(19) United States
(12) Patent Application Publication
(10) Pub. No.: US 2026/0013720 A1
(43) Pub. Date: Jan. 15, 2026

(54) RETINAL IMAGING SYSTEM AND RETINAL IMAGING ADAPTOR AND RELATED METHODS OF USE

(71) Applicant: Verily Life Sciences LLC, Dallas, TX (US)

(72) Inventors: JEREMY EMKEN, Belmont, CA (US); JONATHAN GROSSMAN, San Francisco, CA (US); ERIC BENNETT, San Carlos, CA (US); ETHAN GLASSMAN, Palo Alto, CA (US); SHUN-WEI WU, San Francisco, CA (US); SHIVANKIT SETHI, South San Francisco, CA (US)

(21) Appl. No.: 18/881,958

(22) PCT Filed: Jun. 13, 2023
(86) PCT No.: PCT/US2023/025182

§ 371 Date: Jan. 7, 2025

Related U.S. Application Data
(60) Provisional application No. 63/370,406, filed on Aug. 4, 2022.

(51) Int. Cl. A61B 3/12; A61B 3/00

(52) U.S. Cl. CPC A61B 3/12; A61B 3/0033; A61B 3/0083

(57) ABSTRACT

A retinal imaging system and a retinal imaging adaptor and related methods of use are described. In an embodiment; the retinal imaging system comprises a monocular image sensor adapted to acquire a retinal image of an eye; and a binocular eye piece configured to slide relative to the monocular image sensor and shaped to couple to a face of a user. In an embodiment, the monocular image sensor is positioned to acquire a first retinal image of a first eye of the user in a first position of the binocular eye piece and to acquire a second retinal image of a second eye of the user in a second position of the binocular eye piece. In an embodiment, the retinal imaging system includes a sliding bracket coupled to a housing of the retinal imaging system and slidably coupled to the binocular eye piece.

### CROSS-REFERENCE TO RELATED APPLICATION

This application claims the benefit of U.S. Provisional Application 63/370,406, filed Aug. 4, 2022, which is hereby incorporated by reference in its entirety.

### TECHNICAL FIELD

This disclosure relates generally to retinal imaging technologies, and in particular but not exclusively, relates to illumination techniques for retinal imaging.

### BACKGROUND INFORMATION

Retinal imaging is a part of basic eye exams for screening, field diagnosis, and progress monitoring of many retinal diseases. A high-fidelity retinal image is important for accurate screening, diagnosis, and monitoring.

A conventional retinal camera system uses a single eyebox having a single location, generally defined relative to the eyepiece lens of the conventional retinal camera system, from which both the left-side and right-side eyes are imaged.

However, this single location is a compromise location that is not optimized for the individual eye and furthermore does not account for the need to obtain higher quality images in specific regions of interest within the left and/or right eyes to help the doctor screen, diagnose, monitor, or treat specific ophthalmic pathologies.

One challenge in using such a conventional retinal imaging system is a user has to know which eye is being imaged. Frequently, an eye is imaged twice or the wrong eye is imaged. Such scenarios can require additional imaging and may include return visits for the patient. Complexity and confusion can increase when an operator sits opposite from the patient, thus making less clear which eye is to be imaged and which direction the subject should move to align their eyes with the retinal imaging system.

### BRIEF DESCRIPTION OF THE DRAWINGS

Non-limiting and non-exhaustive embodiments of the invention are described with reference to the following figures, wherein like reference numerals refer to like parts throughout the various views unless otherwise specified. Not all instances of an element are necessarily labeled so as not to clutter the drawings where appropriate. The drawings are not necessarily to scale, emphasis instead being placed upon illustrating the principles being described.

FIG. 1A illustrates a perspective view of a retinal imaging system, according to an embodiment of the present disclosure.

FIG. 1B illustrates a front view of the retinal imaging system of FIG. 1A in a first position, according to an embodiment of the present disclosure.

FIG. 1C illustrates a front view of the retinal imaging system of FIG. 1A in a second position, according to an embodiment of the present disclosure.

FIG. 1D illustrates a top-down plan view of the retinal imaging system of FIG. 1A, according to an embodiment of the present disclosure.

FIG. 1E illustrates a side view of the retinal imaging system of FIG. 1A, according to an embodiment of the present disclosure.

FIG. 2A illustrates a perspective view of a retinal imaging adaptor, according to an embodiment of the present disclosure.

FIG. 2B illustrates a top-down view of the retinal imaging adaptor of FIG. 2A in a first position, according to an embodiment of the present disclosure.

FIG. 2C illustrates another top-down view of the retinal imaging adaptor of FIG. 2A in a second position, according to an embodiment of the present disclosure.

FIG. 2D illustrates a bottom-up view of the retinal imaging adaptor of FIG. 2A, according to an embodiment of the present disclosure.

FIG. 2E illustrates another perspective view of the retinal imaging adaptor of FIG. 2A, according to an embodiment of the present disclosure.

FIG. 2F illustrates a front view of the retinal imaging adaptor of FIG. 2A, according to an embodiment of the present disclosure.

FIG. 3 is a partial exploded illustration of a retinal imaging adaptor, according to an embodiment of the present disclosure.

FIG. 4 schematically illustrates a retinal imaging system, in accordance with an embodiment of the disclosure.

FIG. 5 is a perspective illustration of a retinal imaging system, in accordance with an embodiment of the present disclosure.

FIG. 6 is a flow chart illustrating a process for capturing retinal images based upon eye sidedness, in accordance with an embodiment of the disclosure.

FIGS. 7A & 7B illustrate a dynamic fixation target including an eyebox reference and an eye location reference that coax the eye into a specific eyebox and alignment, in accordance with an embodiment of the disclosure.

### DETAILED DESCRIPTION

Embodiments of a retinal imaging system, a retinal imaging adaptor, and method of operation for a retinal imaging system are described herein. In the following description numerous specific details are set forth to provide a thorough understanding of the embodiments. One skilled in the relevant art will recognize, however, that the techniques described herein can be practiced without one or more of the specific details, or with other methods, components, materials, etc. In other instances, well-known structures, materials, or operations are not shown or described in detail to avoid obscuring certain aspects.

Reference throughout this specification to “one embodiment” or “an embodiment” means that a particular feature, structure, or characteristic described in connection with the embodiment is included in at least one embodiment of the present invention. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

High fidelity retinal images are important for screening, diagnosing, and monitoring many retinal diseases. To this end, acquiring high-fidelity images of both eyes of users having two eyes is desired. Conventional retinal imaging systems may not clearly indicate which eye to image when acquiring retinal images of a user.

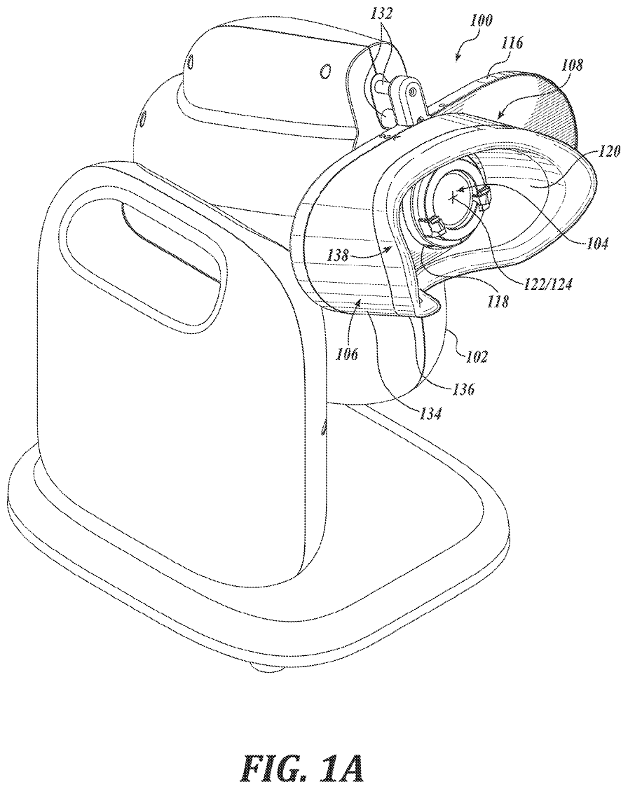

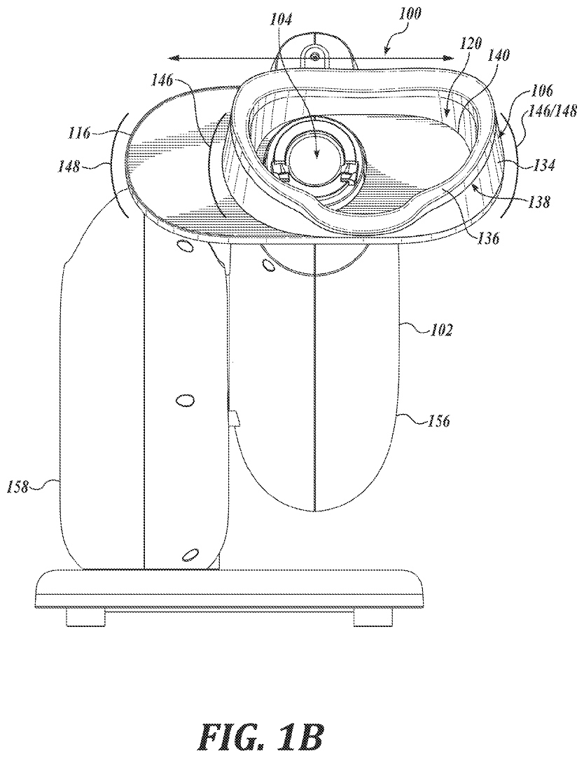

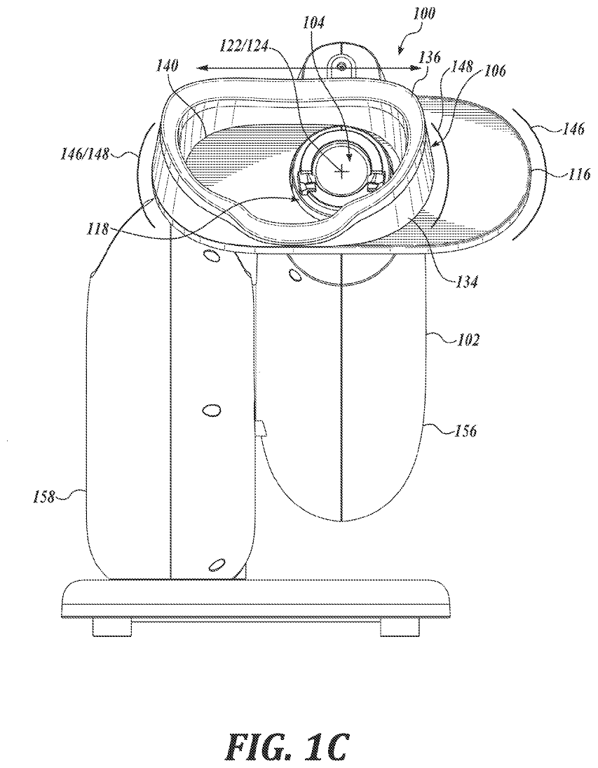

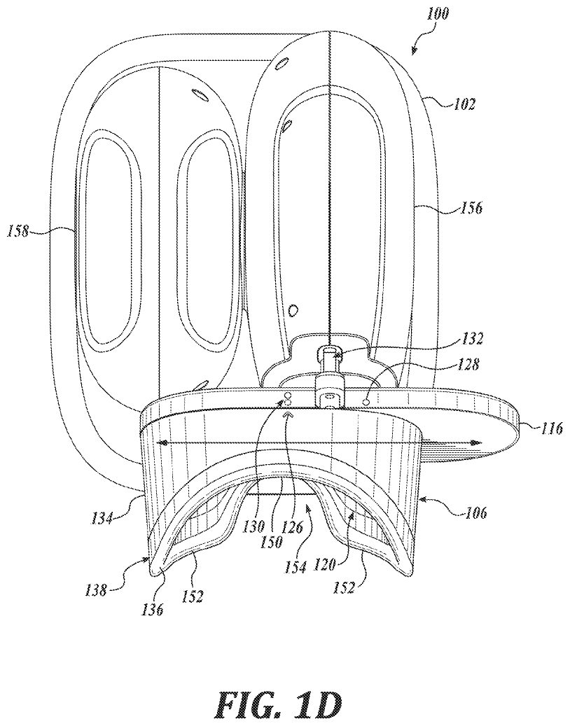

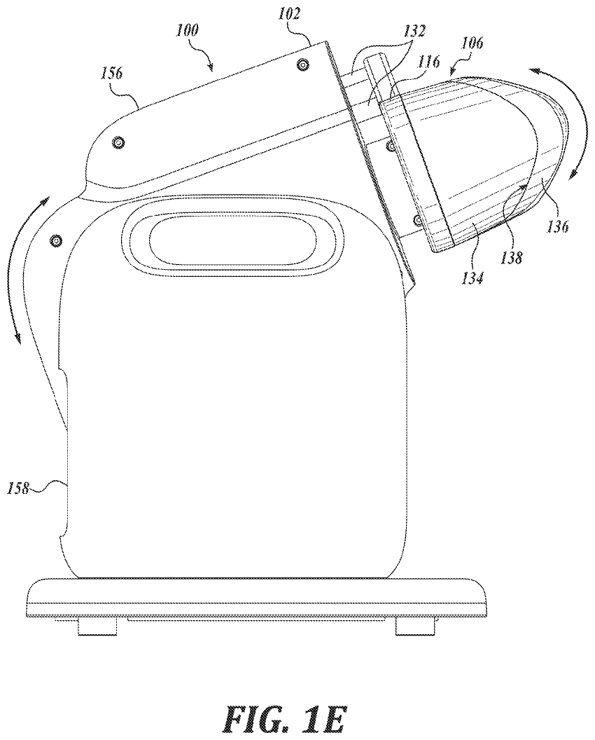

To address these and related needs, the present disclosure provides, in an aspect, a retinal imaging system suitable to selectively image a first eye and a second eye of a user. In this regard, attention is directed to FIGS. 1A-1E in which a retinal imaging system 100, according to an embodiment of the present disclosure, is illustrated.

FIG. 1A illustrates a perspective view of the retinal imaging system 100. FIG. 1B illustrates a front view of the retinal imaging system 100. FIG. 1C illustrates another front view of the retinal imaging system 100. FIG. 1D) illustrates a top-down plan view of the retinal imaging system 100. FIG. 1F illustrates a side view of the retinal imaging system 100.

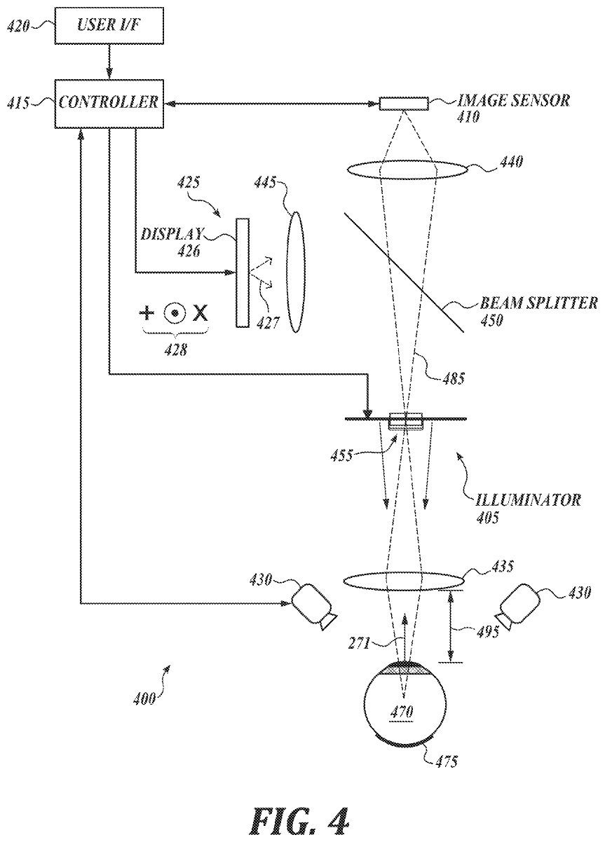

In the illustrated embodiment, the retinal imaging system 100 is shown to include a housing 102 and a monocular image sensor 104 disposed within the housing 102 and adapted to acquire a retinal image of an eye of a user. In an embodiment, the monocular image sensor 104 is configured to acquire a retinal image of a single eye per retinal image. Additional discussion of a retinal image sensor and examples of its component parts and operation, according to embodiments of the present disclosure, is provided further herein with respect to FIG. 4. In an embodiment, the monocular image sensor 104 includes lenses, illuminators, image sensors, and the like discussed further herein with respect to FIG. 4.

As shown, the retinal imaging system 100 further includes a binocular eye piece 106. As discussed further herein, the binocular eye piece 106 is configured to slide relative to the housing 102, such as relative to the monocular image sensor 104, and shaped to couple to a face of a user. In this regard, the monocular image sensor 104 is positioned to acquire a first retinal image of a first eye of the user in a first position of the binocular eye piece 106. See, for example, FIG. 1B. Likewise, the monocular image sensor 104 is positioned to acquire a second retinal image of a second eye of the user in a second position of the binocular eye piece 106. See, for example. FIG. 1C.

The binocular eye piece 106 is shown as a goggle shaped to couple with and conform to a face of the user. While a goggle-like shape is shown, it will be understood that other form factors are possible which allow acquisition of retinal images of both eyes of a patient.

In the illustrated embodiment, retinal imaging system 100 is shown to include a sliding bracket 116 coupled to the housing 102, where the sliding bracket 116 is also slidably coupled to the binocular eye piece 106. The slidable coupling between the sliding bracket 116 and the binocular eyepiece allows the binocular eye piece 106 to slide relative to the monocular imaging sensor between the first position and the second position in order to acquire retinal images of the first and second eye of a user, respectively.

The sliding bracket 116 is shown to define a bracket aperture 118 shaped allow light from an interior portion 120 of the binocular eye piece 106 to pass through the bracket aperture 118 for receipt by the monocular image sensor 104. As illustrated, the bracket aperture 118 overlaps with the monocular image sensor 104. This allows light to pass from the interior portion 120, through the bracket aperture 118 for receipt by the monocular image sensor 104.

In an embodiment, a central axis 122 of the bracket aperture 118 is colinear with an imaging axis 124 of the monocular image sensor 104. In an embodiment, the binocular eye piece 106 is configured to slide orthogonally relative to the imaging axis 124. In an embodiment, such orthogonal sliding relative to the imaging axis 124 allows the binocular eye piece 106 to move from the first position to the second position and vice versa, such as to acquire, with the monocular image sensor 104, retinal images of the first and second eye of the user.

In an embodiment, the binocular eye piece 106 is configured to move relative to the monocular image sensor 104 in a direction generally orthogonal to a median plane, i.e., generally parallel to a coronal plane, of a face of the user when the face is disposed against the binocular eye piece 106.

In an embodiment, the sliding bracket 116 comprises indicia, such as in the form of markings, etchings, indentations, and the like, indicating where the sliding bracket 116 is placed relative to a binocular eye piece 106 marker to achieve different positions of the retinal imaging system 100. In the illustrated embodiment, the binocular eye piece 106 is shown to comprise an eye piece reference marker 126, shown here as a chevron. As also shown, the sliding bracket 116 comprises a first bracket reference marker 128 (shown here as a single dot) aligning with the eye piece reference marker 126 when the binocular eye piece 106 is in the first position; and a second bracket reference marker 130 (shown here as a vertical pair of dots) aligning with the eye piece reference marker 126 when the binocular eye piece 106 is in the second position. While a chevron and dots are shown as markers, it will be understood that other written, printed, embossed, etc. indicia are within the scope of the present disclosure. Alignment of the binocular eye piece 106 reference marker with the first bracket reference marker 128 indicates that the binocular eye piece 106 is in the first position and alignment of the second bracket reference marker 130 indicates that the binocular eye piece 106 is the second position.

The retinal imaging system 100 can include further structures and features indicating alignment of the binocular eye piece 106 relative to the monocular image sensor 104 according to various positions of the retinal imaging system 100. In an embodiment, a curvature 146 of an edge of the binocular eye piece 106 matches a curvature 148 of an edge of the sliding bracket 116. Such a curvature match is configured to provide a visual queue, such as to a user and/or operator of the retinal imaging system 100, that the binocular eye piece 106 is positioned in one of the first position or the second position when the curvature 146 of the edge of the binocular eye piece 106 and the curvature 148 of the edge of the sliding bracket 116 are aligned. In another embodiment, the binocular eye piece 106 and/or the sliding bracket 116 comprise one or more detents, such as one or more spring ball detents, configured to provide to a user an audible and/or tactile indication that the binocular eye piece 106 is in the first position of the second position.

In an embodiment, the binocular eye piece 106 is configured to move relative to the monocular image sensor 104 to place an eye of the user closer or farther away from the monocular image sensor 104. Such relative movement can be useful to adjust placement of the user's eye within an eye box of the monocular image sensor 104 to acquire an appropriate retinal image of the eye. In an embodiment, the sliding bracket 116 is configured to move along an imaging axis 124 of the monocular image sensor 104 relative to the housing 102.

As shown, the retinal imaging system 100 includes at least two stanchions 132 coupled to the sliding bracket 116. In an embodiment, the at least two stanchions 132 are slidably received by the housing 102 and configured to move the sliding bracket 116 relative to the housing 102, such as along the imaging axis 124. In an embodiment, the at least two stanchions 132 are received by the housing 102 with respective linear bearings. By including at least two stanchions 132, rotational movement of the binocular eye piece 106, such as about the imaging axis 124, is limited or eliminated.

In an embodiment, the at least two stanchions 132 are biased by a spring, such as a constant-force spring, to provide resistance to moving the binocular eye piece 106 toward the monocular image sensor 104. In an embodiment, the biasing force of the spring is sufficient to return the binocular eye piece 106 to a resting or starting position after compression and, yet is not so high that it cannot be compressed by the face of a user, such as with the neck muscles of a user. In this regard, a user can adjust the binocular eye piece 106 relative to the monocular image sensor 104 (i.e., place their eye within an eye box of the monocular image sensor 104) and the binocular eye piece 106 will subsequently return to an original position.

As discussed further herein, in certain embodiments, the binocular eye piece 106 is configured to conform to the face of a user, such as when portions of the face are pressed or otherwise disposed against the binocular eye piece 106. In an embodiment, the binocular eye piece 106 comprises a frame 134 slidably coupled to the sliding bracket 116; and a compressible edge cushion 136 disposed on an eye-facing edge 138 of the frame 134 and positioned to contact the face of the user. The compressible edge cushion 136 is configured to provide comfort to the user. The compressible edge cushion 136 also allows a user to move their face, and in particular their eyes, relative to the binocular eye piece 106 while maintaining contact with the binocular eye piece 106 and in this regard, to limit light leaking into the interior portion 120 of the binocular eye piece 106 from between the binocular eye piece 106 and the face of the user.

In the illustrated embodiment, the binocular eye piece 106 comprises a forehead rest 150 shaped and positioned to contact a forehead of the user when the face of the user is disposed against the binocular eyepiece. As shown, the binocular eye piece 106 also includes a cheek rest 152 shaped and positioned to contact a cheek of the user when the face of the user is disposed against the binocular eye piece 106. In an embodiment, the forehead rest 150 and the cheek rest 152 are shaped and positioned to induce a downward pitch of a head of the user when the forehead and the cheek of the user are disposed against the binocular eye piece 106. In this regard, the binocular eye piece 106 including the forehead rest 150 and the check rest 152 is configured to induce an opening of the palpebral fissure height (the visible portion of the eye) which facilitates eye tracking by minimizing eye lid interference.

In an embodiment, the binocular eye piece 106 is shaped to provide space for the nose of the user, such as when the face is disposed against the binocular eye piece 106 and when a user is adjusting their eyes relative to the monocular image sensor 104 to achieve alignment between the eye and the monocular image sensor 104. In this regard, the binocular eye piece 106 defines a cutout 154 shaped to receive a nose of the user when the face of the user is disposed against the binocular eye piece 106 and to allow movement of the nose within the cutout 154.

In an embodiment, the binocular eye piece 106 is shaped or otherwise configured to limit light entering an interior portion 120 of the binocular eye piece 106 and the face of the user when the face of the user is disposed against the compressible edge cushion 136. In this regard, in an embodiment, the frame 134 is opaque to visible and other light, such as to limit light leakage into the interior portion 120. In an embodiment, the frame 134 has a matte finish and/or a dark color configured to reduce light reflection and absorb stray light, respectively, that might degrade retinal image quality.

As shown, the frame 134 defines a frame aperture 140 shaped and positioned to overlap with the bracket aperture 118 when the sliding bracket 116 is coupled to the binocular eye piece 106 in both the first position and the second position. In this regard, the frame 134 is configured to provide a line of sight between the monocular image sensor 104 and first and second eyes of the user.

In an embodiment, the retinal imaging system 100 is configured to adjust an angle of the binocular eye piece 106 relative to the face of the user. In particular, in an embodiment, the retinal imaging system 100 is configured to adjust an angle of the binocular eye piece 106 relative to a canthal line of the user, such as when the face of the user is disposed against the binocular eye piece 106. In this regard the retinal imaging system 100 is configured to align the eyes of the user with an optical pathway of the monocular image sensor 104, such as without adjusting a height of the retinal imaging system 100. Accordingly, as shown, the housing 102 comprises a first portion 156 carrying the monocular image sensor 104 and a second portion 158 rotatably coupled to the first portion 156. In the illustrated embodiment, the second portion 158 is configured to be disposed on a resting surface, such as a tabletop or desk. While a desktop retinal imaging system 100 is illustrated, it will be understood that the retinal imaging system 100 can be configured to rest on a floor, hang from a ceiling, move up and down a separate structure, and the like. By rotating the first portion 156 relative to the second portion 158, the monocular image sensor 104 can be selectively aligned with a user's canthal line and/or align the eyes of the user with the monocular image sensor 104.

In another aspect, the present disclosure provides a retinal imaging adaptor. In an embodiment, the retinal imaging adaptor is shaped or otherwise configured to couple with a retinal imaging system for acquiring a retinal image of eyes of a user. In an embodiment, the retinal imaging adaptor is an example of a portion of the retinal imaging system 100 discussed further herein with respect to FIGS. 1A-1E.

In an embodiment, the retinal imaging adaptor comprises a binocular eye piece shaped to couple to a face of a user; and a sliding bracket configured to couple to a retinal imaging system and slidably coupled to the binocular eye piece, wherein the sliding bracket defines a bracket aperture shaped allow light from an interior portion of the binocular eye piece to pass through the bracket aperture.

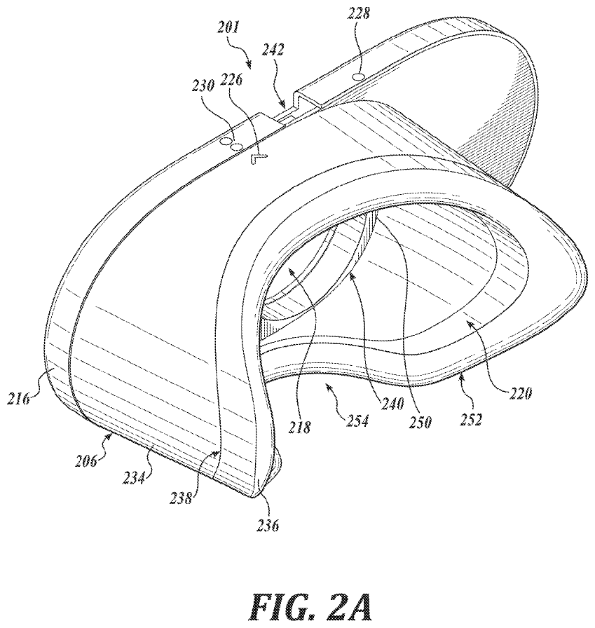

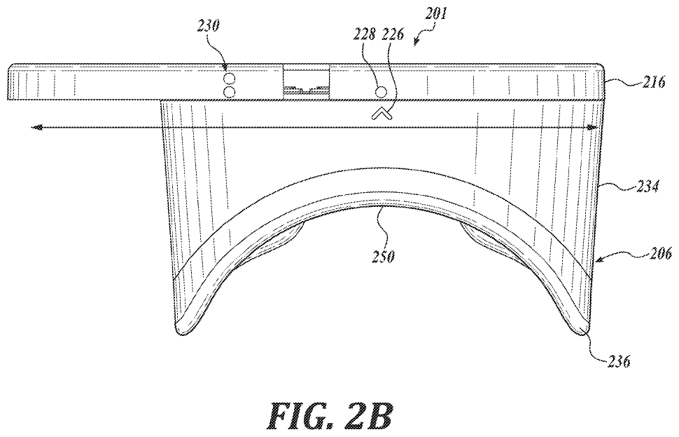

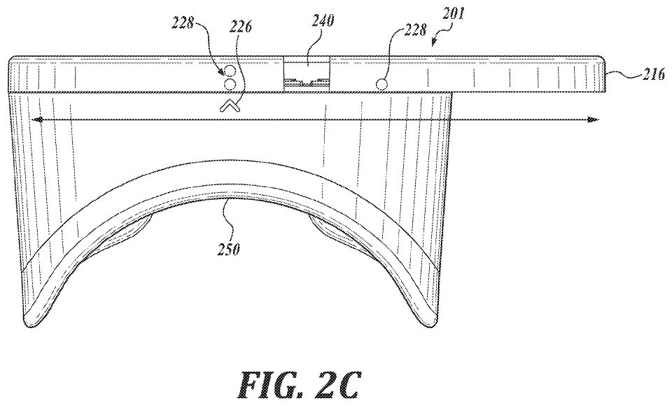

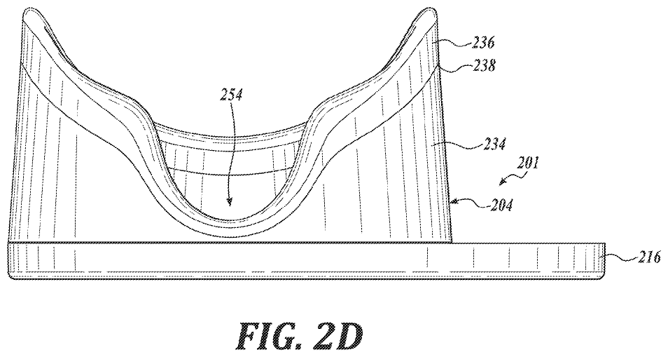

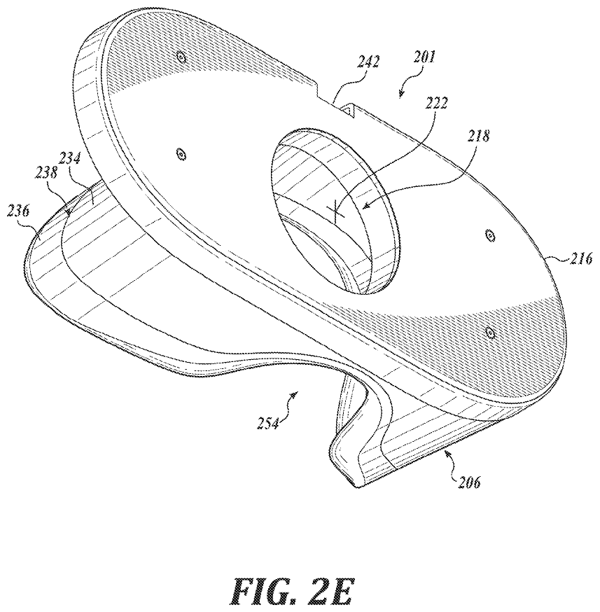

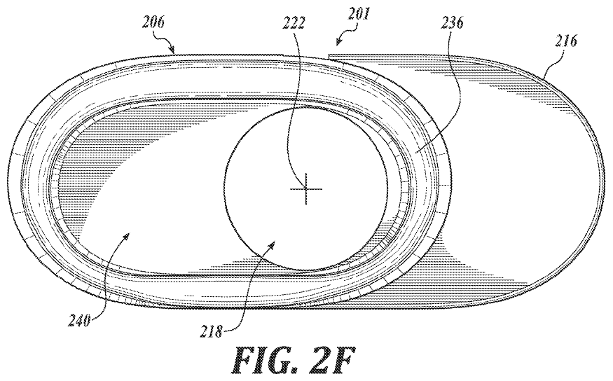

In this regard, attention is directed to FIGS. 2A-2F in which a retinal imaging adaptor 201 according to an embodiment of the present disclosure is illustrated. FIG. 2A illustrates a perspective view of a retinal imaging adaptor 201. FIG. 2B illustrates a top-down view of the retinal imaging adaptor 201. FIG. 2C illustrates another top-down view of the retinal imaging adaptor 201. FIG. 2D illustrates a bottom-up view of the retinal imaging adaptor 201. FIG. 2E illustrates another perspective view of the retinal imaging adaptor 201. FIG. 2F illustrates a front view of the retinal imaging adaptor 201.

As shown, the retinal imaging adaptor 201 includes a binocular eye piece 206 shaped to couple to a face of a user. In an embodiment, the binocular eye piece 206 is an example of the binocular eye piece 106 discussed further herein with respect to FIGS. 1A-1E.

In illustrated embodiment, the retinal imaging adaptor 201 further includes a sliding bracket 216 configured to couple, such as fixedly couple, to a retinal imaging system and slidably coupled to the binocular eye piece 206. As shown, the sliding bracket 216 defines a bracket aperture 218 shaped allow light from an interior portion 220 of the binocular eye piece 206 to pass through the bracket aperture 218. In the illustrated embodiment, a central axis 222 of the bracket aperture is position to align with an imaging axis (not shown, see FIGS. 1A-1E) of a retinal imaging system coupled to the retinal imaging adaptor 201.

In this regard, the bracket aperture 218 is shaped and positioned to allow light from within the interior portion 220 of the binocular eye piece 206, such as light emitted from within an eye of a user, to pass through the bracket aperture 218 and onto, for example, a monocular image sensor of a retinal imaging system coupled to the retinal imaging adaptor 201.

As illustrated, the binocular eye piece 206 is configured to slide relative to the sliding bracket 216. In this regard, as the binocular eye piece 206 slides relative to the sliding bracket 216 coupled thereto, the bracket aperture 218 is positioned to allow light from a first eye of the user to pass through the bracket aperture 218 in a first position of the binocular eye piece 206 and to allow light from a second eye of the user to pass through the bracket aperture 218 in a second position of the binocular eye piece 206.

In the illustrated embodiment, the binocular eye piece 206 comprises a frame 234 slidably coupled to the sliding bracket 216. In an embodiment, the frame 234 is configured to limit light entering an interior portion 220 of the frame 234 from an exterior environment, such as to improve retinal image quality. In an embodiment, the frame 234 is opaque to light, such as visible, infrared, or ultraviolet light. In an embodiment, the frame 234 has a matte finish configured to limit light reflection. In an embodiment, the frame 234 has a dark color, such as black, configured to absorb a broad spectrum of light incident upon the frame 234.

The binocular eye piece 206 is also shown to include a compressible edge cushion 236 disposed on an eye-facing edge 238 of the frame 234 and positioned to contact the face of the user. In an embodiment, the compressible edge cushion 236 is configured to compress and/or conform to the face of a user when disposed against the compressible edge cushion 236. In this regard, the binocular eye piece 206 is shaped to limit light entering an interior portion 220 of the binocular eye piece 206 and the face of the user when the face of the user is disposed against the compressible edge cushion 236.

As shown, the binocular eye piece 206 also comprises or otherwise defines a forehead rest 250 shaped and positioned to contact a forehead of the user when the face of the user is disposed against the binocular eyepiece; and a cheek rest 252 shaped and positioned to contact a cheek of the user when the face of the user is disposed against the binocular eye piece 206. In this regard, the binocular eye piece 206 is shaped to receive the face of the user disposed against the binocular eye piece 206, such as to acquire retinal imaging.

In the illustrated embodiment, the binocular eye piece 206, and, in particular the frame 234 and compressible edge cushion 236, defines a cutout 254 shaped to receive a nose of the user when the face of the user is disposed against the binocular eye piece 206 and to allow movement of the nose within the cutout 254. As discussed further herein with respect to FIGS. 1A-1E, the cutout 254 allows a user to rotate or otherwise adjust their head to align an eye with imaging optics of a monocular image sensor of a retinal imaging system.

In an embodiment, the frame 234 defines a frame aperture 240 shaped to overlap with the bracket aperture 218 when the sliding bracket 216 is coupled to the binocular eye piece 206 in both the first position and the second position. In this regard, the frame aperture 240 is shaped to allow light from the interior portion 220 of the binocular eye piece 206 to exit the interior portion 220 through the bracket aperture 218, such as for receipt by a monocular image sensor of a retinal imaging system coupled to the retinal imaging adaptor 201.

As shown, the binocular eye piece 206 comprises an eye piece reference marker 226. The sliding bracket 216 is shown to include a first bracket reference marker 228 aligning with the eye piece reference marker 226 when the binocular eye piece 206 is in the first position; and a second bracket reference marker 230 aligning with the eye piece reference marker 226 when the binocular eye piece 206 is in the second position. As discussed further herein with respect to FIGS. 1A-1E, the eye piece reference marker 226 and the first bracket reference marker 228 and the second bracket reference marker 230 are configured to aid a user in aligning the binocular eye piece 206 with the sliding bracket 216 into a first position and a second position, respectively, such as to acquire a retinal image of a first eye and a second eye of a user.

As above, the retinal imaging adaptor 201 is shaped or otherwise configured to couple to a retinal imaging system, such as a housing of the retinal imaging system. In this regard, in an embodiment, the retinal imaging adaptor 201 comprises a mounting attachment 242, such as disposed in the sliding bracket 216. configured to releasably attach to a housing of the retinal imaging system.

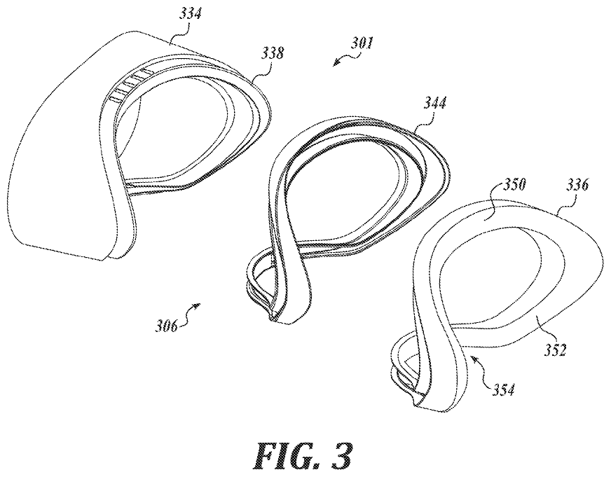

FIG. 3 is a partial exploded illustration of a retinal imaging adaptor 301, according to an embodiment of the present disclosure. In an embodiment, the portions of the retinal imaging adaptor 301 are portions of the retinal imaging adaptor 201 discussed further herein with respect to FIGS. 2A-2E. In an embodiment, the portions of the retinal imaging adaptor 301 are portions of the binocular eye piece 106 discussed further herein with respect to FIGS. 1A-1E.

The illustrated embodiment illustrates a binocular eye piece 306. As shown, the binocular eye piece 306 comprises a frame 33; and a compressible edge cushion 336 disposed on an eye-facing edge 338 of the frame 334 and positioned to contact the face of the user. In an embodiment, the compressible edge cushion 336 comprises a soft, compressible material configured to conform to a face of a user disposed against the compressible edge cushion 336.

The binocular eye piece 306 is shown to further include molding 344 shaped to couple, on a first side, to the frame 334, and, on a second side, to the compressible edge cushion 336.

The illustrated binocular eye piece 306 is configured to be slidably coupled to a sliding bracket, such as sliding brackets 116 and/or 216 discussed further herein with respect to FIGS. 1A-1E and 2A-2E.

As shown, the binocular eye piece comprises and/or defines a forehead rest 350 shaped and positioned to contact a forehead of the user when the face of the user is disposed against the binocular eyepiece; and a check rest 352 shaped and positioned to contact a check of the user when the face of the user is disposed against the binocular eye piece.

The binocular eye piece is also shown to define a cutout 354 shaped to receive a nose of the user when the face of the user is disposed against the binocular eye piece and to allow movement of the nose within the cutout 354.

FIG. 4 illustrates a retinal imaging system 400, in accordance with an embodiment of the disclosure. The illustrated embodiment of retinal imaging system 400 includes an illuminator 405, an image sensor 410 (also referred to as a retinal image sensor), a controller 415, a user interface 420, a dynamic fixation target 425, an alignment tracking camera system 430, and an optical relay system. The illustrated embodiment of the optical relay system includes lens assemblies 435, 440, 445 and a beam splitter 450. Lens assembly 435 may also be referred to as an eyepiece lens assembly 435. The illustrated embodiment of illuminator 405 comprises a dynamic ring illuminator with a center aperture 455. The illustrated embodiment of dynamic fixation target 425 includes a display 426 that outputs a dynamic fixation image 427 that may include one or more reference markers 428 that represent relative eye box and/or eye locations.

The optical relay system serves to direct (e.g., pass or reflect) illumination light 480 output from illuminator 405 along an illumination path through the pupil of eye 470 to illuminate retina 475 while also directing image light 485 of retina 475 (i.e., the retinal image) along an imaging path to image sensor 410. Image light 485 is formed by the scattered reflection of illumination light 480 off of retina 475. In the illustrated embodiment, the optical relay system further includes beam splitter 450, which passes at least a portion of image light 485 to image sensor 410 while also optically coupling dynamic fixation target 425 to eyepiece lens assembly 435 and directing dynamic fixation image 427 output from display 426 to eye 470. Beam splitter 450 may be implemented as a polarized beam splitter, a non-polarized beam splitter (e.g., 90% transmissive and 10% reflective, 50/50 beam splitter, etc.), a multi-layer dichroic beam splitter, or otherwise. The optical relay system includes a number of lenses, such as lenses 435, 440, and 445, to focus the various light paths as needed. For example, lens 435 may include one or more lensing elements that collectively form an eyepiece lens assembly that is displaced from the cornea of eye 470 by an eye relief 495 during operation. Lens 440 may include one or more lens elements for bring image light 485 to a focus on image sensor 410. Lens 445 may include one or more lens elements for focusing dynamic fixation image 427. It should be appreciated that optical relay system may be implemented with a number and variety of optical elements (e.g., lenses, reflective surfaces, diffractive surfaces, etc.) and may vary from the configuration illustrated in FIG. 4.

In one embodiment, dynamic fixation image 427 output from display 426 represents a point of fixation upon which the patient can accommodate their focus and fix their gaze. The dynamic fixation image 427 may be an image of a plus-sign, a bullseye, a cross, a target, circles, or other shape or collection of shapes. In the illustrated embodiment, dynamic fixation target 425 is implemented as virtual images output from a display 426. However, the point of fixation may be implemented in a variety of other ways including physical target(s) that are actuated or optically manipulated. Dynamic fixation target 425 not only can aid with obtaining alignment between retinal imaging system 400 and eye 470 by providing visual feedback to the patient, but may also give the patient a fixation point/target upon which the patient can accommodate and stabilize their vision. The dynamic fixation target may be moved by translating the image of the fixation target (e.g., reference markers 428) about display 426 as desired (e.g., moving a symbol or image up/down or left/right on display 426). Display 426 may be implemented with a variety of technologies including a liquid crystal display (LCD), light emitting diodes (LEDs), various illuminated shapes (e.g., an illuminated cross or concentric circles), or otherwise. Of course, the dynamic fixation target may be implemented in other manners than a virtual image on a display. For example, the dynamic fixation target may be a physical object (e.g., crosshairs, etc.) that is physically manipulated.

Controller 415 is coupled to image sensor 410, display 426, illuminator 405, and alignment tracking camera system 430 to orchestrate their operation. Controller 415 may include software/firmware logic executing on a microcontroller, hardware logic (e.g., application specific integrated circuit, field programmable gate array, etc.), or a combination of software and hardware logic. Although FIG. 4 illustrates controller 415 as a distinct functional element, the logical functions performed by controller 415 may be decentralized across a number hardware elements. Controller 415 may further include input/output (I/O)) ports, communication systems, or otherwise. Controller 415 is coupled to user interface 420 to receive user input and provide user control over retinal imaging system 400. User interface 420 may include one or more buttons, dials, joysticks, feedback displays, indicator lights, etc.

Image sensor 410 may be implemented using a variety of imaging technologies, such as complementary metal-oxide-semiconductor (CMOS) image sensors, charged-coupled device (CCD) image sensors, or otherwise. In one embodiment, image sensor 410 includes an onboard memory buffer or attached memory to store/buffer retinal images. In one embodiment, image sensor 410 may include an integrated image signal processor (ISP) to permit highspeed digital processing of retinal images buffered in the onboard memory. The onboard image buffer and ISP may facilitate high frame rate image burst captures, image processing, image stacking, and output of high-quality composite retinal images. The integrated ISP may be considered a decentralized component of controller 415.

Alignment tracking camera system 430 operates to track lateral alignment (or misalignment) and relief offset between retinal imaging system 400 and eye 470, and, in particular, between eyepiece lens assembly 435 and eye 470. System 430 may operate using a variety of different techniques to track the relative position of eye 470 to retinal imaging system 400 including pupil tracking or iris tracking. In the illustrated embodiment, system 430 includes two cameras disposed on either side of eyepiece lens assembly 435 to enable triangulation and obtain X, Y, and Z gross position information about the pupil or iris. In one embodiment, system 430 also includes one or more infrared (IR) emitters to track eye 470 with IR light while retinal images are acquired with bursts of visible spectrum light output through eyepiece lens assembly 435 from illuminator 405. In such an embodiment, IR filters may be positioned within the image path to filter the IR tracking light. In some embodiments, the tracking illumination is temporally offset from image acquisition with white light bursts.

Lateral eye alignment may be measured via retinal images acquired by image sensor 410, or separately/additionally, by system 430. In the illustrated embodiment, system 430 is positioned externally to view eye 470 from outside of eyepiece lens assembly 435. In other embodiments, system 430 may be optically coupled via the optical relay components to view and track eye 470 through eyepiece lens assembly 435.

During operation, controller 415 operates illuminator 405 and retinal image sensor 410 to capture one or more retinal images. Illumination light 480 is directed through the pupil of eye 470 to illuminate retina 475. The scattered reflections from retina 475 are directed back along the image path through aperture 455 to image sensor 410. When eye 470 is properly aligned within the selected eye box of system 400, aperture 455 operates to block deleterious reflections and light scattering that would otherwise malign the retinal image while passing the image light itself. Prior to capturing the retinal image, controller 415 operates display 426 to output a dynamic fixation image 427 to guide the patient's gaze. One or more initial or preliminary eye images (e.g., initial alignment images), either from image sensor 410 or alignment tracking camera system 430, are acquired and analyzed to determine the lateral alignment between eye 470 and eyepiece lens assembly 435. These initial alignment images may be illuminated with infrared (IR) light output from illuminator 405 (or an independent illuminator associated with alignment tracking camera system 430) so as not to trigger an iris constriction response, which narrows the imaging path to retina 475. In other embodiments, conventional white light or other chromatic light is used to acquire the initial alignment images. The initial alignment image is then analyzed by controller 415 to identify any misalignment, reposition an eye location reference within dynamic fixation image 427 to encourage appropriate eye positioning relative to the selected eye box, and then trigger acquisition of one or more subsequent eye images (e.g., retinal image burst) with image sensor 410. The subsequent images may be full color images, specific chromatic images, or even IR images as desired.

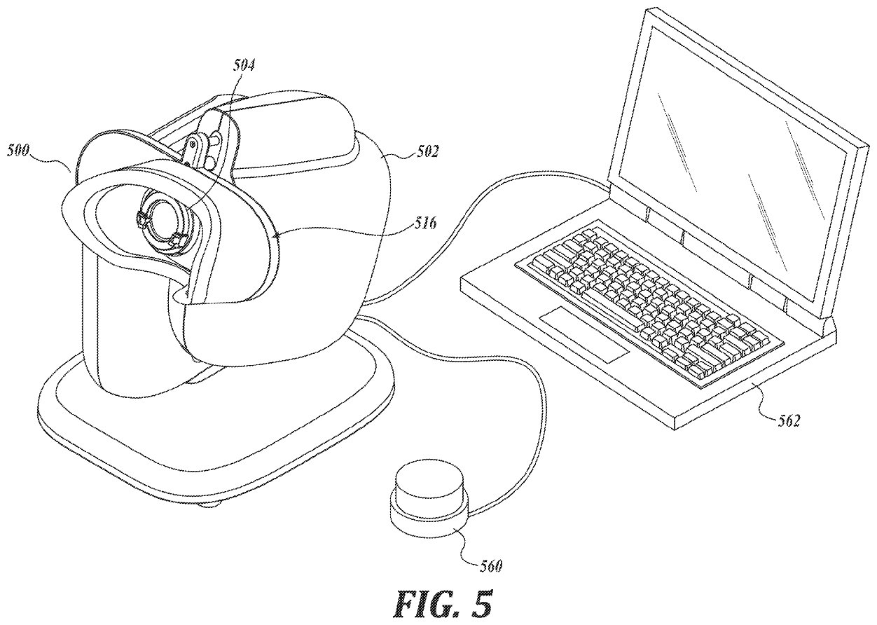

FIG. 5 is a perspective illustration of a retinal imaging system 500, in accordance with an embodiment of the present disclosure. As shown, the retinal imaging system 500 is shown to include a monocular image sensor 504 disposed in a housing 502, and a binocular eye piece 516 slidably coupled to the housing 502. In an embodiment, the monocular image sensor 504, the housing 502, and the binocular eye piece 516 are examples of the monocular image sensor 104, the housing 102, and the binocular eye piece 116 discussed further herein with respect to FIGS. 1A-1E.

The retinal imaging system 500 is shown to further include a focusing nob 560 configured to selectively focus an image viewable with an eye of a user when a face of the user is disposed against the binocular eye piece 516, such as during retinal imaging of the eye.

In the illustrated embodiment, the retinal imaging system 500 includes a user interface 562, shown here as a laptop computer. While a laptop computer is shown, it will be understood that other user interfaces 562, such as tablets, smartphones, touchscreens, and the like, are within the scope of the present disclosure. In an embodiment, user interface 562 is an example of user interface 420 discussed further herein with respect to FIG. 4. In an embodiment, user interface 562 is configured to receive user input and provide user control over retinal imaging system 562.

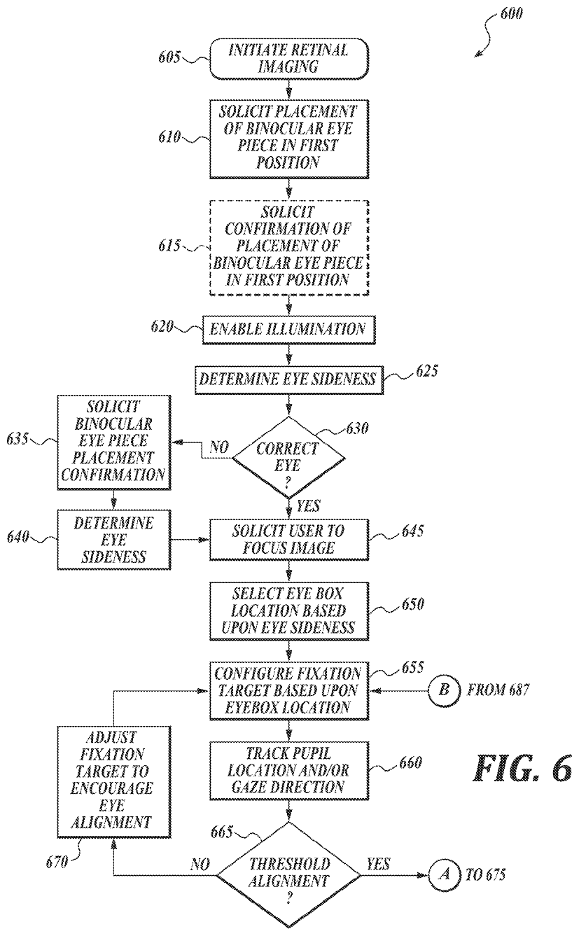

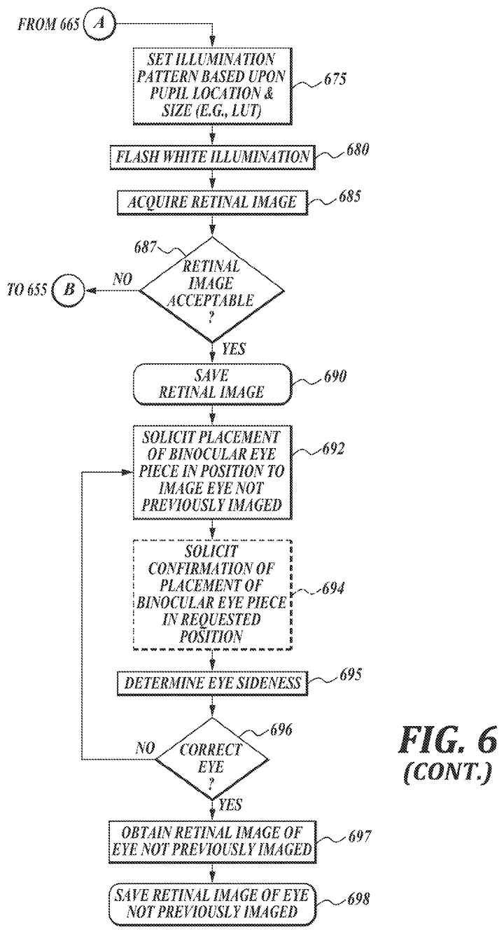

FIG. 6 is a flow chart illustrating a process 600 for capturing retinal images based upon eye sidedness, in accordance with an embodiment of the disclosure. The order in which some or all of the process blocks appear in process 600 should not be deemed limiting. Rather, one of ordinary skill in the art having the benefit of the present disclosure will understand that some of the process blocks may be executed in a variety of orders not illustrated, or even in parallel.

In a process block 605, the retinal imaging process is initiated. Initiation may include the user pressing a power button on user interface 420. After powering on, the retinal imaging system 400 may solicit placement of the binocular eye piece, such as binocular eye piece 516, to acquire a retinal image of a first eye, as part of process block 610.

In a process block 615, the user interface 420 may solicit confirmation of placement of the binocular eye piece 516 in a first position. Soliciting such confirmation from a user and/or operator has been found to increase a likelihood that the binocular eye piece 516 is placed in the correct position, such as to acquire a retinal image of the first eye.

In process block 620, illumination is enabled to obtain preliminary eye images to facilitate eye tracking and/or determine eye-sidedness. In one embodiment, this initial illumination is IR illumination output from alignment tracking camera system 430.

In a process block 625, the eye-sidedness (i.e., right-sided eye or a left-sided eye) is determined. Eye-sidedness may be manually input via user interface 420 or automatically determined by controller 415 based upon image analysis and feature identification performed on a preliminary image of the eye. The preliminary image may be an IR retinal image acquired via image sensor 410 and/or eye images acquired by alignment tracking camera system 430.

If it appears that an eye not corresponding to the first position of the binocular eye piece is being imaged in the preliminary image as determined in decision block 630, the user interface 420 will solicit confirmation of placement of the binocular eye piece in the first position, as part of process block 635. Subsequently, eye sidedness is determined again in process block 640.

Regardless of which eye is in position to be imaged following process blocks 640 or 635, the user will be solicited to focus an image (process block 645), such as displayed by display 426, and with focusing knob 560.

With eye-sidedness determined (process blocks 625-640), the eyebox location for retinal imaging system 400 may be selected (process block 650). The eyebox location is the location of the eyebox of the retinal imaging system, which is a bound region in space defined relative to the eyepiece lens assembly. The eyebox location for the right eye will generally be offset to the left (e.g., offset left approximately 1.5 mm) while the eyebox location for the left eye will generally be offset to the right (e.g., offset right approximately 1.5 mm).

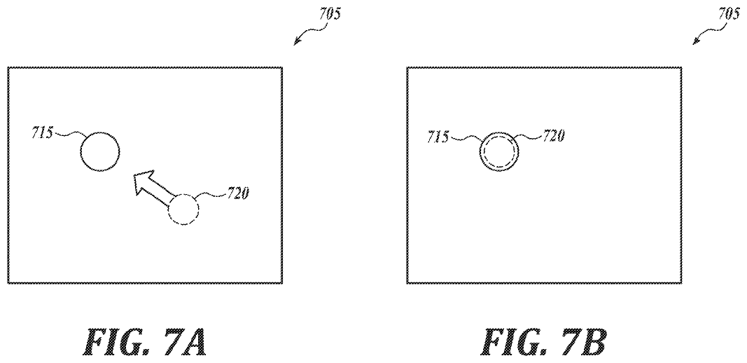

With the eyebox location selected, the fixation location of dynamic fixation target 425 may be configured to encourage eye 470 to adjust its position and/or gaze direction accordingly (process block 655). FIGS. 7A and 7B illustrate example dynamic fixation images 705 and 710, respectively, output from display 426. The dynamic fixation images 705 and 710 both include an eyebox reference 715 and an eye location reference 720. Eyebox reference 715 is a virtual marker on display 426 that is positioned on display 426 based upon the selected eyebox location and represents the eyebox itself. Eye location reference 720 is a virtual marker on display 426 that represents the patient's pupil and its position on display 426 changes in real-time as the user attempts to align their eye with eyepiece lens 435. In other words, the position of eye location reference 720 tracks the eye location relative to eyepiece lens 435 based upon output from alignment tracking camera system 430 or image sensor 410 (process block 660). In various embodiments, alignment tracking camera system 430 may be used for gross eye alignment based upon pupil/iris tracking while image sensor 410 may be used for fine eye alignment based upon retinal tracking. Dynamic fixation images 705 and 710 may operate as a sort of game where the patient is told to concentrically align the two circular markers by moving their eye relative to eyepiece lens assembly 435. Fyebox alignment is achieved when the eye location reference 720 is moved into eyebox reference 714 (decision block 665), as illustrated in FIG. 7B. By dynamically moving the eye location reference 720, the fixation target is adjusted, and the user is encouraged or coaxed into alignment (process block 670) as they attempt to concentrically align the reference markers. Of course, other alignment/fixation images may be implemented to encourage the necessary threshold alignment for obtaining a satisfactory retinal image.

As threshold alignment is achieved (decision block 665), illuminator 405 is configured by controller 415 to select the appropriate illumination pattern for retinal imaging. The illumination pattern may be selected based upon pupil location and pupil size to reduce image artifacts and optimize retinal image quality (process block 670). In one embodiment, a lookup table (LUT) may index illumination patterns to pupil position and/or pupil size. In yet other embodiments, the LUT may further index illumination patterns to POI and/or eye sidedness for further pattern refinement. For example, the illumination pattern may not only consider the current location of the eye relative to eyepiece lens assembly 435, but also the anatomical feature that is relevant to a given pathology and thus select an illumination pattern that shifts various image artifacts away from that anatomic feature in the retinal images. This may be considered a finer illumination pattern refinement in addition to the selection of the illumination pattern based upon real-time eye position tracking.

With threshold alignment achieved (decision block 665) and the appropriate illumination pattern selected (process block 675), illuminator 405 illuminates retina 475 through the pupil. This illumination may be a white light flash, though the particular wavelengths used for illumination (e.g., broadband white light, IR light, near-IR, etc.) may be tailored for a particular pathology or application. The illumination flash in process block 680 may only last for a period of time (e.g., 200 msec) that is less than or equal to the human physiological response time (e.g., pupil constriction or eye blink). While illumination is active, one or more retinal images are acquired (process block 685). In one embodiment, acquisition of a burst of retinal images (e.g., 5, 10, 20, 50, 100 images) is triggered during the illumination window and while the eye remains in the selected eyebox as determined from real-time feedback from alignment tracking camera system 430 (or image sensor 410).

The burst of retinal images may be buffered onboard a camera chip including image sensor 410 where an image signal processor (ISP) can quickly analyze the quality of the acquired retinal images. The ISP may be considered a component of controller 415 (e.g., decentralized offload compute engine) that is located close to image sensor 410 to enable highspeed image processing. If the images are occluded, obscured, or otherwise inadequate, then process 600 returns to process block 650 to repeat the relevant portions of process 600. However, if the acquired images are collectively deemed sufficient to adequately capture an image of the retina, then the retinal image(s) may be saved to provide a high-quality retinal image (process block 675).

Following saving the retinal image of the first imaged eye, the retinal imaging system may initiate acquiring, etc., a retinal image of the eye not previously imaged. In this regard, the retinal imaging system may proceed with process blocks 610-690, but in obtaining a retinal image of the eye that was not image previously. More particularly, the retinal image system may solicit placement of the binocular eye piece, such as with the user interface 426, in position to image an eye that was not previously imaged (process block 692). As above, the method 600 solicits placement of the binocular eye piece in a first position (process block 610); however, the method 600 accounts for the binocular eye piece being positioned in a second position and/or imaging a second eye, rather than a first eye, in the process of acquiring a first retinal image. In other words, it is possible that the first retinal image is a retinal image of a second eye, rather than a first eye. In this regard, process block 692 solicits placing the binocular eye piece in position to image the eye that was not previously imaged, which may be either the first or second position of the binocular eye piece depending upon which eye was imaged previously.

Following solicitation to place the binocular eye piece in position to image the eye not previously imaged, the retinal imaging system may solicit confirmation of placement of binocular eye piece in the requested position (i.e., to acquire a retinal image of the eye not previously imaged) in process block 694.

After soliciting placement of the binocular eye piece in position to image an eye not previously imaged (process block 692) and/or soliciting confirmation of placement of binocular eye piece in position in the requested position (process block 694), the retinal imaging system determines eye sidedness (i.e., which eye is being imaged) as part of process block 695.

If is determined in decision block 696 that the eye being imaged is the eye that was previously imaged, the process of soliciting and confirming placement of the binocular eye piece (process blocks 692 and 694) and determining eye sidedness is repeated until it is determined that the eye not previously imaged is positioned to acquire a retinal image. In this regard, for patients having two eyes, the method 600 is configured to obtain retinal images for both eyes.

Once the eye not previously imaged is positioned for retinal imaging, the method 600 includes acquiring a retinal image of the eye not previously imaged, as in process block 697.

If the retinal image of the eye not previously imaged is acceptable, the method 600 includes saving the retinal image of the eye not previously imaged as in process block 698.

Certain processes explained above are described in terms of computer software and hardware. The techniques described may constitute machine-executable instructions embodied within a tangible or non-transitory machine (e.g., computer) readable storage medium, that when executed by a machine will cause the machine to perform the operations described. Additionally, the processes may be embodied within hardware, such as an application specific integrated circuit (“ASIC”) or otherwise.

A tangible machine-readable storage medium includes any mechanism that provides (i.e., stores) information in a non-transitory form accessible by a machine (e.g., a computer, network device, personal digital assistant, manufacturing tool, any device with a set of one or more processors, etc.). For example, a machine-readable storage medium includes recordable/non-recordable media (e.g., read only memory (ROM), random access memory (RAM), magnetic disk storage media, optical storage media, flash memory devices, etc.).

The above description of illustrated embodiments of the invention, including what is described in the Abstract, is not intended to be exhaustive or to limit the invention to the precise forms disclosed. While specific embodiments of, and examples for, the invention are described herein for illustrative purposes, various modifications are possible within the scope of the invention, as those skilled in the relevant art will recognize.

These modifications can be made to the invention in light of the above detailed description. The terms used in the following claims should not be construed to limit the invention to the specific embodiments disclosed in the specification. Rather, the scope of the invention is to be determined entirely by the following claims, which are to be construed in accordance with established doctrines of claim interpretation.

What is claimed is:

1. A retinal imaging system comprising:
a monocular image sensor adapted to acquire a retinal image of an eye; and
a binocular eye piece configured to slide relative to the monocular image sensor and shaped to couple to a face of a user, wherein the monocular image sensor is positioned to acquire a first retinal image of a first eye of the user in a first position of the binocular eye piece and to acquire a second retinal image of a second eye of the user in a second position of the binocular eye piece.

2. The retinal imaging system of claim 1, further comprising:
a sliding bracket coupled to a housing of the retinal imaging system and slidably coupled to the binocular eye piece,
wherein the sliding bracket defines a bracket aperture shaped allow light from an interior portion of the binocular eye piece to pass through the bracket aperture for receipt by the monocular image sensor.

3. The retinal imaging system of claim 2, wherein the bracket aperture overlaps with the monocular image sensor.

4. The retinal imaging system of claim 2, wherein the binocular eye piece comprises an eye piece reference marker, and wherein the sliding bracket comprises:
a first bracket reference marker aligning with the eye piece reference marker when the binocular eye piece is in the first position; and
a second bracket reference marker aligning with the eye piece reference marker when the binocular eye piece is in the second position.

5. The retinal imaging system of claim 2, wherein the sliding bracket is configured to move along an imaging axis of the monocular image sensor relative to the monocular image sensor.

6. The retinal imaging system of claim 5, further comprising:
at least two stanchions coupled to the sliding bracket,
wherein the at least two stanchions are slidably received by the housing and configured to move the sliding bracket relative to the housing along the imaging axis.

7. The retinal imaging system of claim 2, wherein the binocular eye piece comprises:
a frame slidably coupled to the sliding bracket; and
a compressible edge cushion disposed on an eye-facing edge of the frame and positioned to contact the face of the user.

8. The retinal imaging system of claim 7, wherein the frame defines a frame aperture shaped to overlap with the bracket aperture when the sliding bracket is coupled to the binocular eye piece in both the first position and the second position.

9. The retinal imaging system of claim 7, wherein the binocular eye piece is shaped to conform to at least a portion of the face of the user, thereby limiting light entering the interior portion of the binocular eye piece when the face of the user is disposed against the compressible edge cushion.

10. The retinal imaging system of claim 2, wherein a curvature of an edge of the binocular eye piece matches a curvature of an edge of the sliding bracket.

11. The retinal imaging system of claim 2, wherein the housing comprises:
a first portion carrying the monocular image sensor; and
a second portion rotatably coupled to the first portion.

12. The retinal imaging system of claim 1, wherein the retinal imaging system further comprises:
a user interface configured to receive user input; and
a controller communicatively coupled to the monocular image sensor and the user interface, the controller including logic that when executed causes the retinal imaging system to perform operations including:
soliciting, with the user interface, placement of the binocular eye piece in the first position;
determining a sidedness of a first eye of a subject;
acquiring, with the monocular image sensor, a retinal image the first eye;
soliciting, with the user interface, placement of the binocular eye piece in a position to acquire a retinal image of a second eye of the subject; and
acquiring, with the monocular image sensor, a retinal image the second eye.

13. The retinal imaging system of claim 1, wherein the binocular eye piece comprises:
a forehead rest shaped and positioned to contact a forehead of the user when the face of the user is disposed against the binocular eyepiece; and
a cheek rest shaped and positioned to contact a cheek of the user when the face of the user is disposed against the binocular eye piece,
wherein the forehead rest and the cheek rest are shaped and positioned to induce a downward pitch of a head of the user when the forehead and the cheek of the user are disposed against the binocular eye piece.

14. The retinal imaging system of claim 1, wherein the binocular eye piece defines a cutout shaped to receive a nose of the user when the face of the user is disposed against the binocular eye piece and to allow movement of the nose within the cutout.

15. A retinal imaging adaptor comprising:
a binocular eye piece shaped to couple to a face of a user; and
a sliding bracket configured to couple to a retinal imaging system and slidably coupled to the binocular eye piece,
wherein the sliding bracket defines a bracket aperture shaped allow light from an interior portion of the binocular eye piece to pass through the bracket aperture.

16. The binocular eye piece of claim 15, wherein the bracket aperture is positioned to allow light from a first eye of the user to pass through the bracket aperture in a first position of the binocular eye piece and to allow light from a second eye of the user to pass through the bracket aperture in a second position of the binocular eye piece.

17. The binocular eyepiece of claim 15, wherein the sliding bracket comprises a mounting attachment configured to releaseably attach to a housing of the retinal imaging system.

18. The binocular eyepiece of claim 15, wherein the binocular eye piece comprises:
a forehead rest shaped and positioned to contact a forehead of the user when the face of the user is disposed against the binocular eyepiece; and
a cheek rest shaped and positioned to contact a cheek of the user when the face of the user is disposed against the binocular eye piece,
wherein the forehead rest and the cheek rest are shaped and positioned to induce a downward pitch of a head of the user when the forehead and the cheek of the user are disposed against the binocular eye piece.

19. The binocular eyepiece of claim 15, wherein the binocular eye piece defines a cutout shaped to receive a nose of the user when the face of the user is disposed against the binocular eye piece and to allow movement of the nose within the cutout.

20. A method of imaging retinas of a subject with a retinal imaging system, the method comprising:
soliciting, with a user interface of the retinal imaging system, placement of a binocular eye piece of the retinal imaging system in a first position;
determining a sidedness of a first eye of the subject;
acquiring, with a monocular image sensor of the retinal imaging system, a retinal image the first eye;
soliciting, with the user interface, placement of the binocular eye piece in a position to acquire a retinal image of a second eye of the subject; and
acquiring, with the monocular image sensor, a retinal image of the second eye.
